(19) United States
(12) Plant Patent Application Publication
(10) Pub. No.: US 2009/0172855 P1
(43) Pub. Date: Jul. 2, 2009

(54) Poinsettia plant named 'NPCW07129'

(50) ;

(76) Inventor: Nils Klemm, Stuttgart (DE)

Correspondence Address: JONDLE & ASSOCIATES P.C., 858 HAPPY CANYON ROAD SUITE 230, CASTLE ROCK, CO, 80108, US

(21) Appl. No.: 12/005,213

(22) Filed: Dec. 26, 2007

(51) Int. Cl. A01H 5/00

(52) U.S. Cl. PLT/304

(57) ABSTRACT

A new Poinsettia plant particularly distinguished by its red and white bi-colored bracts and compact plant habit is disclosed.

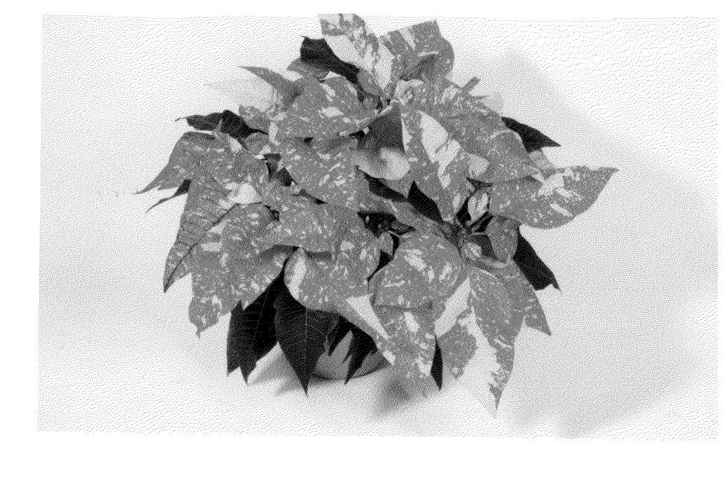

### GENUS AND SPECIES

Euphorbia pulcherrima

### VARIETY DENOMINATION

‘NPCW07129’

### BACKGROUND OF THE NEW PLANT

The present invention comprises a new and distinct cultivar of poinsettia plant, botanically known as Euphorbia pulcherrima, and hereinafter referred to by the cultivar name ‘NPCW07129’. The new cultivar is the result of an induced mutation in the poinsettia plant ‘Christmas Dream Pink’ (unpatented) made in the year 2004 in Stuttgart, Germany.

Plants of ‘Christmas Dream Pink’ were irradiated with x-rays in Stuttgart, Germany in 2004. The treated plants were grown in Stuttgart, Germany, and were propagated by shoot tip cuttings. The resulting plants were screened for positive mutations and examined during the flowering period in winter 2004/2005. Among these plants, a red and white bi-colored plant was selected and asexually multiplied for further examination in April 2005.

The new cultivar was created in 2004 in Stuttgart, Germany and has been asexually reproduced repeatedly by vegetative cuttings in Stuttgart, Germany over 10 generations. ‘NPCW07129’ has not been observed under all possible environmental conditions. The present invention has been found to retain its distinctive characteristics through successive asexual propagations.

### SUMMARY OF THE INVENTION

The following are the most outstanding and distinguishing characteristics of this new cultivar when grown under normal horticultural practices in Stuttgart, Germany.
1. Red and white bi-color bracts; and
2. A compact growth habit.

### DESCRIPTION OF PHOTOGRAPH

This new poinsettia plant is illustrated by the accompanying photograph which is an overhead view of the plant and shows bracts, flower buds, and the foliage of the plant; the colors shown are as true as can be reasonably obtained by conventional photographic procedures. The photograph is of a whole plant about 3 months old grown in a greenhouse in Stuttgart, Germany, in November of 2007.

### DESCRIPTION OF THE NEW CULTIVAR

The following detailed descriptions set forth the distinctive characteristics of ‘NPCW07129’. The data which define these characteristics were collected from asexual reproductions carried out in Stuttgart, Germany. The plant history was taken on 4-month-old plants which were planted as rooted cuttings in 12-cm pots and were pinched in week 33. The plants were grown in a greenhouse under natural light. Color readings were taken under natural light in the greenhouse. Color references are primarily to the R.H.S. Colour Chart of The Royal Horticultural Society of London (R.H.S.) (2001).

### DETAILED BOTANICAL DESCRIPTION OF THE NEW PLANT

Classification:
   Family.—Euphorbiaceae.
   Botanical.—Euphorbia pulcherrima (Willd. Ex Klotzsch).
   Common name.—Poinsettia.
Parentage: X-ray induced mutation of the red-colored poinsettia plant ‘Christmas Dream Pink’ (unpatented)
Growth:
   Form.—Shrub, self-branching, triangular.
   Growth and branching habit.—Upright, compact growth habit.
   Height (from soil line to the top).—20.0 cm.
   Width.—25.0 cm.
   Time to produce a rooted cutting.—About 20 days.
   Time to produce a finished flowering plant.—About 55 days.
   Keeping quality.—About 25 days.
Branches:
   Average number.—4 to 6.
   Length.—18.0 cm.
   Internode length.—1.0 cm to 3.0 cm.
   Diameter.—0.5 cm to 1.0 cm.
   Stem color (upper and lower parts).—RHS 143A.
Leaves:
   Quantity.—8 to 12 per branch.
   Arrangement.—Alternate.
   Size.—Length: 4.0 cm to 10.0 cm Width: 2.0 cm to 7.0 cm.
   Shape.—Ovate.
   Margin.—Serrate.
   Apex.—Acute.
   Base.—Obtuse.
   Lobes.—Slightly lobed.
   Color (mature leaves).—Upper surface: RHS 139A Lower surface: RHS 137B.
   Color (immature leaves).—Upper surface: RHS 144B Lower surface: RHS 143C.
   Texture (both surfaces).—Smooth.
   Venation color.—Upper surface: RHS 139A Lower surface: RHS 139D.
   Variegation.—None.
   Leaf petiole.—Length: 2.0 cm to 4.0 cm Diameter: 0.2 cm to 0.3 cm Color (both surfaces): Upper surface: RHS 181B Texture (both surfaces): Smooth Aspect: Horizontally directed, leaf blades slanting somewhat downward.
Bracts:
   Number per inflorescence.—6 to 8.
   Shape.—Ovate.
   Apex.—Obtuse.
   Base.—Obtuse.
   Lobes.—Slightly lobed.
   Size.—Length: 7.0 cm to 10.0 cm Diameter: 4.0 cm to 6.0 cm.
   Texture (both surfaces).—Smooth.
   Bract color (both surfaces).—RHS 47B with RHS 5D spots and blotches.
   Vein color (both surfaces).—RHS 47B with RHS 5D spots and blotches.
   Bract petiole.—Length: 1.0 cm to 3.0 cm Diameter: 0.2 cm Color (both surfaces): RHS 47A.
Inflorescence:
   Blooming habit.—Intermittent.
   Inflorescence type.—Medium size, flat with the bracts mostly horizontally directed, in the center a tight cyme with medium size cyathia.
   Average number of inflorescences.—4 to 6.
   Lastingness of the inflorescences on the plant.—About 4 to 5 weeks with no dropping of bracts.
   Diameter.—15.0 cm to 20.0 cm.
   Height.—2.0 cm to 5.0 cm.
Cyme:
   Cyme diameter.—1.5 cm to 2.5 cm.
   Cyathia number.—4 to 7 per inflorescence.
   Cyathium.—Shape: Ovate Diameter: 0.3 cm to 0.5 cm Length: 0.4 cm to 0.7 cm Color: RHS 143C.
   Peduncle.—Color: RHS 144C Length: 0.1 cm to 0.2 cm.
   Nectar cups.—Number: One per cyathium Length: 0.2 cm to 0.3 cm Color: RHS 13A and RHS 14C.
Reproductive organs:
   Stamens.—Number: 5 to 10 per cyathium Shape: Ovate Filaments: Color: RHS 47B with RHS 5D spots Length: 0.25 cm Pollen: Quantity: Sparse Color: RHS 9A to RHS 12A.
   Gynoecium.—Present.
Fruit and seed set: No seed set observed so far
Disease and insect resistance: Disease and insect resistance is typical of the species

### COMPARISON WITH PARENTAL AND COMMERCIAL CULTIVARS

‘NPCW07129’ differs from the parental cultivar ‘Christmas Dream Pink’ (unpatented) by having red and white bi-colored bracts instead of pink bracts. In addition, ‘NPCW07129’ has lower vigor than ‘Christmas Dream Pink’.

‘NPCW07129’ differs from the commercial cultivar ‘Primero White Glitter’ (unpatented), by having more compact growth habit, fewer white spots and thinner stems than ‘Primero White Glitter’.

I claim:

1. A new and distinct cultivar of Poinsettia plant as shown and described herein.
